(19) United States
(12) Patent Application Publication
(10) Pub. No.: US 2023/0110482 A1
(43) Pub. Date: Apr. 13, 2023

(54) ADJUSTABLE LENGTH STENT

(71) Applicant: BOSTON SCIENTIFIC SCIMED, INC., Maple Grove, MN (US)

(72) Inventors: Martyn G. Folan, Galway (IE); Gary Gilmartin, Foxford (IE)

(73) Assignee: BOSTON SCIENTIFIC SCIMED, INC., Maple Grove, MN (US)

(21) Appl. No.: 17/963,797

(22) Filed: Oct. 11, 2022

Related U.S. Application Data
(60) Provisional application No. 63/254,683, filed on Oct. 12, 2021.

(51) Int. Cl. A61F 2/966

(52) U.S. Cl. CPC A61F 2/966; A61F 2002/9511

(57) ABSTRACT

A stent configured to change in length while maintaining a constant inner diameter is disclosed. The stent includes an elongated tubular member comprising at least one knitted filament forming a plurality of twisted knit stitches with rungs extending circumferentially between adjacent twisted knit stitches, wherein each twisted knit stitch is interconnected with a longitudinally adjacent twisted knit stitch forming a series of linked stitches. The tubular member is configured to be stretched from a first length to a second length while in a radially expanded configuration without substantially changing the inner and/or outer diameter of the tubular member.

### CROSS-REFERENCE TO RELATED APPLICATIONS

This application claims the benefit of U.S. Provisional Patent Application Ser. No. 63/254,683, filed Oct. 12, 2021, which is incorporated herein by reference.

### TECHNICAL FIELD

The present disclosure pertains to medical devices, methods for manufacturing medical devices, and uses thereof. More particularly, the present disclosure pertains to a stent for implantation in a body lumen, and associated methods.

### BACKGROUND

Implantable medical devices (e.g., expandable stents) may be designed to treat a variety of medical conditions in the body. For example, some expandable stents may be designed to radially expand and support a body lumen and/or provide a fluid pathway for digested material, blood, or other fluid to flow therethrough following a medical procedure. Some medical devices may include radially or self-expanding stents which may be implanted transluminally via a variety of medical device delivery systems. These stents may be implanted in a variety of body lumens such as coronary or peripheral arteries, the esophageal tract, gastrointestinal tract (including the intestine, stomach and the colon), tracheobronchial tract, urinary tract, biliary tract, vascular system, etc.

In some instances it may be desirable to design stents to include sufficient flexibility and elongation properties while maintaining sufficient radial force and diameter to open the body lumen at the treatment site. However, in some stents, the elongation, compressible and flexible properties that assist in stent delivery may also result in a stent that reduces in diameter and tends to migrate from its originally deployed position. For example, stents to be positioned in the gastrointestinal tract must maintain a desired diameter and be resistant to kinking when bent, particularly at angles of 90 degrees or more. Additionally, the generally moist and inherently lubricious environment of the digestive and biliary tracts further contributes to a stent's tendency to migrate when deployed therein.

Therefore, in some instances it may be desirable to design a stent with the ability to elongate while maintaining a constant diameter and to resist kinking when bending. Examples of medical devices including such features are disclosed herein.

### SUMMARY

This disclosure provides design, material, manufacturing method, and use alternatives for medical devices. An example stent configured to change in length while maintaining a constant inner diameter includes a tubular member having a proximal end, a distal end, and a longitudinal axis extending therebetween, the tubular member comprising a knitted filament forming a plurality of twisted knit stitches with rungs extending circumferentially between adjacent twisted knit stitches, wherein each twisted knit stitch is interconnected with a longitudinally adjacent twisted knit stitch forming a series of linked stitches, the tubular member configured to automatically radially expand from a constrained configuration during delivery to a radially expanded configuration, wherein when in the radially expanded configuration, the tubular member has a first length and a first inner diameter and is configured to be stretched to an elongated configuration having a second length and a second inner diameter, and wherein the first length is shorter than the second length and the first and second inner diameters are substantially the same.

Alternatively or additionally to the embodiment above, the second length is at least 200% or more of the first length.

Alternatively or additionally to any of the embodiments above, the first length is about 50 mm to about 60 mm and the second length is about 100 mm to about 160 mm.

Alternatively or additionally to any of the embodiments above, when in the radially expanded configurations the series of linked stitches defines a helix, the helix having a first angle relative to the longitudinal axis when the tubular member is at the first length and a second angle relative to the longitudinal axis when the tubular member is at the second length, wherein the first angle is larger than the second angle.

Alternatively or additionally to any of the embodiments above, when in the constrained configuration for delivery, the series of linked stitches defines longitudinal columns.

Alternatively or additionally to any of the embodiments above, each of the plurality of twisted knit stitches includes a loop portion and a crossed base region.

Alternatively or additionally to any of the embodiments above, each of the plurality of twisted knit stitches is formed by a single filament defining the loop portion and the crossed base region.

Alternatively or additionally to any of the embodiments above, the loop portion of at least some of the twisted knit stitches is wrapped around the crossed base region of the longitudinally adjacent twisted knit stitch.

Alternatively or additionally to any of the embodiments above, the stent further comprises a first suture threaded through at least some of the twisted knit stitches at the distal end and a second suture threaded through at least some of the twisted knit stitches at the proximal end of the tubular member.

An example stent assembly includes a stent having a proximal end, a distal end, and a longitudinal axis extending therebetween, the stent comprising a knitted filament forming a plurality of twisted knit stitches with rungs extending circumferentially between radially adjacent twisted knit stitches, wherein each twisted knit stitch is interconnected with a longitudinally adjacent twisted knit stitch forming a series of linked stitches, the stent configured to automatically radially expand from a constrained configuration during delivery to a radially expanded configuration, wherein when in the radially expanded configuration, the stent has a first length and a first inner diameter and is configured to be stretched to an elongated configuration having a second length and a second inner diameter, wherein the first length is shorter than the second length and the first and second inner diameters are substantially the same, and a delivery device including an outer sleeve and an inner shaft slidable within the outer sleeve, the inner shaft having a distal tip and at least one capture element, wherein the at least one capture element is configured to move between a first configuration positioned adjacent the inner shaft when constrained within the outer sleeve, and a second configuration extending radially outward from the inner shaft when released from the outer sleeve.

Alternatively or additionally to any of the embodiments above, the at least one capture element includes at least one hook.

Alternatively or additionally to any of the embodiments above, the at least one hook includes a first distally facing hook and a second proximally facing hook.

Alternatively or additionally to any of the embodiments above, the at least one capture element includes a plurality of distally facing hooks and a plurality of proximally facing hooks.

Alternatively or additionally to any of the embodiments above, the at least one hook includes a first hook coupled to a second hook.

Alternatively or additionally to any of the embodiments above, the first hook extends through an opening in the second hook.

Alternatively or additionally to any of the embodiments above, the at least one capture element is biased in the second configuration.

Alternatively or additionally to any of the embodiments above, the at least one capture element extends radially 5 mm or more from an outer surface of the inner shaft in the second configuration.

An example method of supporting a body lumen at a stricture includes delivering a stent within the body lumen with a central region of the stent disposed across the stricture, radially expanding the stent to a radially expanded configuration in the body lumen, wherein the stent includes a tubular member having a distal end and a proximal end and a longitudinal axis extending therebetween, the tubular member comprising a knitted filament forming a plurality of twisted knit stitches with rungs extending circumferentially between adjacent twisted knit stitches, wherein each twisted knit stitch is interconnected with a longitudinally adjacent twisted knit stitch forming a series of linked stitches, the tubular member having a first length and a first inner diameter in the radially expanded configuration, and thereafter, stretching the stent within the body lumen to a radially expanded and elongated configuration having a second length, wherein the second length is greater than the first length.

Alternatively or additionally to any of the embodiments above, the stent has a second inner diameter in the radially expanded and elongated configuration, wherein the second inner diameter is substantially the same as the first inner diameter.

Alternatively or additionally to any of the embodiments above, the second length is at least 200% or more of the first length.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative stent;

FIG. 2 is an enlarged top view of a portion of the illustrative stent of FIG. 1;

FIG. 3 is an illustration of the stent of FIG. 1 in a collapsed configuration;

FIG. 4 is an enlarged view of a portion of the stent of FIG. 3;

FIG. 5 is an enlarged side view of a longitudinal edge of the illustrative stent of FIG. 1;

FIG. 6 is an illustration of a portion of the stent of FIG. 1 disposed within a body lumen;

FIGS. 7A and 7B are side views of the stent of FIG. 1 in expanded and elongated configurations, respectively;

FIGS. 8A-8E illustrate the stent of FIG. 1 being deployed and elongated within the biliary tract;

FIG. 9 is a side view of the stent of FIG. 1 showing conformability;

FIG. 10 illustrates the stent of FIG. 1 deployed and elongated within the colon;

FIGS. 11A and 11B are side cross-sectional views of an illustrative stent deployment system;

FIG. 12 is a side cross-sectional view of a portion of another stent deployment system; and

FIG. 13 is a side cross-sectional view of a portion of another stent deployment system.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

A variety of self-expanding and balloon-expandable stents are available. Currently available braided and knitted stents offer good radial strength with minimal foreshortening which is desired for esophageal tracheo-bronchial, biliary, and colonic applications. However, the currently available stents often lack the desired degree of conformability for some anatomical applications. For example, braided stents do not tend to conform to bends in the anatomy and instead tend to straighten the vessel or lumen in which they are placed.

Additionally, currently available braided and knitted stents are often manufactured to span a specified diameter and length. For example, 20 mm diameter stents may be available in lengths of 60 mm, 80 mm, 100 mm, 120 mm, and 150 mm. Stents with other diameters may also be provided in a similar number of lengths. This variety of sizes of stents may create increased operational overhead based on the need for specific tooling to cover many stent sizes, increased clinical storage requirements for individual stent sizes often without regard to how popular or requested a particular size may be. As the matrix of available stent sizes is large, often physicians and/or hospitals will purchase select sizes. For example, a hospital might stock large, medium and small stent sizes for a particular application, with the physicians choosing from this reduced matrix when treating their patients. This may result in a stricture that would ideally require a different sized stent being treated with a larger or smaller device because it is available. This may occur in hospitals in order to reduce the costs of stocking the entire matrix of stent sizes and may result in less than desirable results when the physician selects a stent size based on availability rather than the specific needs of the patient and procedure.

An example where stent size selection is of particular importance is in biliary applications. The biliary tree has many side ducts and/or branches which the physician generally wishes to avoid blocking with a stent. At the same time, the physician requires the stent to be long enough to fully span and relieve the stricture with the ends of the stent appropriately positioned, such as for the proximal end of the stent to protrude through the ampulla into the duodenum while the distal end of the stent is positioned distal of the stricture. Correct sizing of the stent in such a procedure is important to the successful outcome of the procedure.

An alternative knitted self-expanding stent is desired that is capable of delivery via a coaxial delivery system to a torturous anatomical bend or other anatomical location, having similar conformability, radial forces, and foreshortening as previous parallel knitted stent configurations, but resists migration and kinking. While the embodiments disclosed herein are discussed with reference to biliary and intestinal stents, it is contemplated that the stents described herein may be used and sized for use in other locations such as, but not limited to: bodily tissue, bodily organs, vascular lumens, non-vascular lumens and combinations thereof, such as, but not limited to, in the coronary or peripheral vasculature, trachea, bronchi, urinary tract, prostate, brain, stomach and the like.

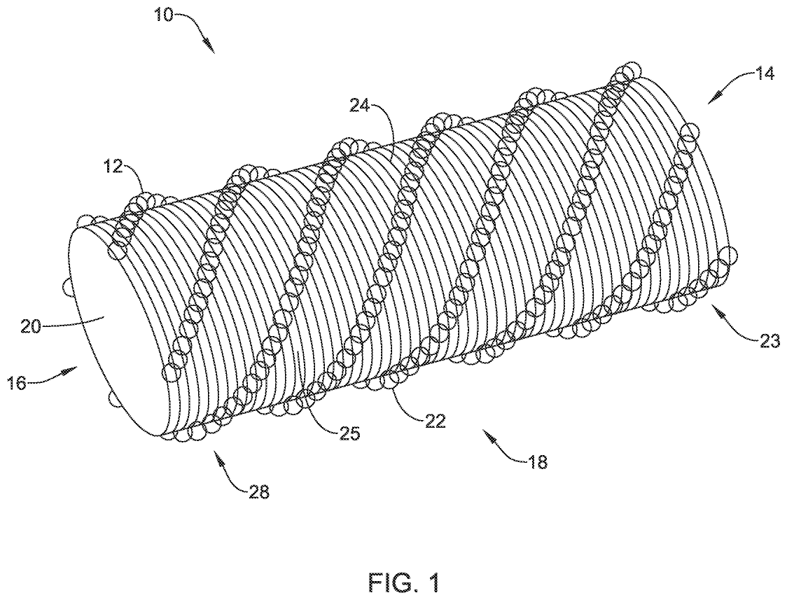

FIG. 1 illustrates a perspective view of an example endoluminal implant, such as, but not limited to, a stent 10. The stent 10 is illustrated with an opaque interior only to more clearly show the structure of the stent without the opposite side making the drawing unclear. It will be understood that in general, no inner structure is present unless otherwise indicated. In some instances, the stent 10 may be formed as a tubular member 12. While the stent 10 is described as generally tubular, it is contemplated that the stent 10 may take any cross-sectional shape desired. The stent 10 may have a first, or distal end 14, a second, or proximal end 16, and a central region 18 disposed between the distal end 14 and the proximal end 16. The stent 10 may include a lumen 20 extending from a first opening adjacent the distal end 14 to a second opening adjacent to the proximal end 16 to allow for the passage of fluids, etc.

The stent 10 may be fabricated from at least one filament 24 defining open cells 25 and twisted knit stitches 22. In some examples, the stent 10 may be formed from only a single filament 24 intertwined with itself to form open cells 25 and twisted knit stitches 22. In some cases, the filament 24 may be a monofilament, while in other cases the filament 24 may be two or more filaments wound, braided, or woven together. In some instances, an inner and/or outer surface of the stent 10 may be entirely, substantially or partially, covered with a polymeric covering or coating. The covering or coating may extend across and/or occlude one or more, or a plurality of the open cells 25 and twisted knit stitches 22 defined by the filament 24. The covering or coating may help reduce tissue ingrowth.

It is contemplated that the stent 10 can be made from a number of different materials such as, but not limited to, metals, metal alloys, shape memory alloys and/or polymers, as desired, enabling the stent 10 to be expanded into shape when accurately positioned within the body. In some instances, the material may be selected to enable the stent 10 to be removed with relative ease as well. For example, the stent 10 can be formed from alloys such as, but not limited to, Nitinol and Elgiloy®. Depending on the material selected for construction, the stent 10 may be self-expanding (i.e., configured to automatically radially expand when unconstrained). In some embodiments, fibers may be used to make the stent 10, which may be composite fibers, for example, having an outer shell made of Nitinol having a platinum core. It is further contemplated the stent 10 may be formed from polymers including, but not limited to, polyethylene terephthalate (PET). In some instances, the filaments of the stent 10, or portions thereof, may be bioabsorbable or biodegradable, while in other instances the filaments of the stent 10, or portions thereof, may be biostable. The stent 10 may be self-expanding. As used herein the term “self-expanding” refers to the tendency of the stent to return to a preprogrammed diameter when unrestrained from an external biasing force (for example, but not limited to a delivery catheter or sheath). In some instances, in the expanded configuration as shown in FIG. 1, the stent 10 may include a first end region 23 adjacent the distal end 14 and a second end region 28 adjacent the proximal end 16.

In some embodiments, the stent 10 may have a uniform outer diameter from the distal end 14 to the proximal end 16 when in the relaxed, expanded configuration, as shown in FIG. 1. In some embodiments, the first end region 23 and the second end region 28 may include retention features or anti-migration flared regions (not explicitly shown) having enlarged diameters relative to the central region 18. Anti-migration flared regions, which may be positioned adjacent to the distal end 14 and the proximal end 16 of the stent 10, may be configured to engage an interior portion of the walls of the esophagus or other body lumen. It is contemplated that a transition from the cross-sectional area of the central region 18 to the retention features or flared regions may be gradual, sloped, or occur in an abrupt step-wise manner, as desired. In some embodiments, the outer diameter of the central region 18 may be in the range of 6 to 14 millimeters. The outer diameter of the anti-migration flares (distal end 14 and/or proximal end 16) may be in the range of 8 to 18 millimeters. It is contemplated that the outer diameter of the stent 10 may be varied to suit the desired application.

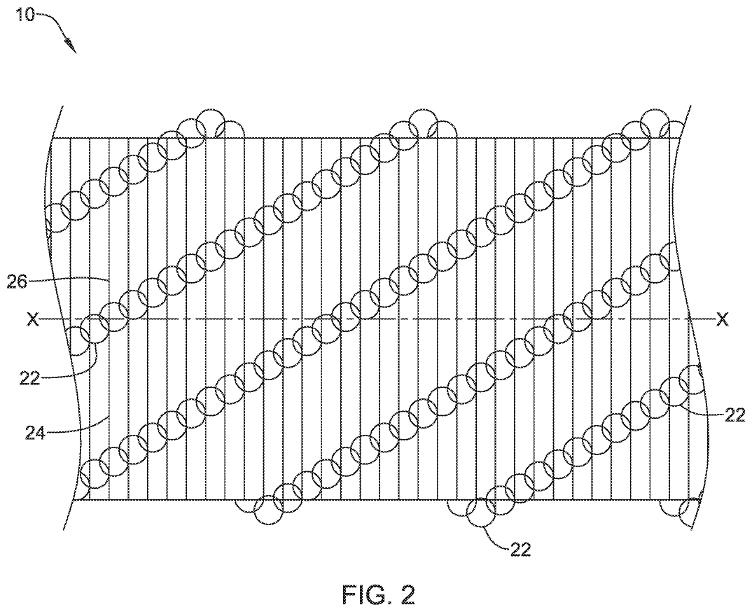

FIG. 2 illustrates the helical structure of the stent 10 when in the radially expanded configuration after release from a delivery catheter. The stent 10 as illustrated may be fabricated from a single knitted filament 24 forming twisted knit stitches 22 separated by elongate rungs 26 extending circumferentially between adjacent twisted knit stitches 22. Each twisted knit stitch 22 may be interconnected with a longitudinally adjacent twisted knit stitch 22 forming a series of linked stitches that extend helically around the stent in the radially expanded configuration, as shown in FIG. 2. The linked twisted knit stitches 22 may define a helix that extends helically around the stent 10 along the entire length of the stent 10. In some embodiments, when the stent 10 is in a fully radially expanded and relaxed state, the rungs 26 may extend substantially perpendicular to the longitudinal axis x-x of the stent 10, as shown in FIG. 2. In some embodiments, the rungs 26 may be between 0.1 mm and 10.0 mm in length in the expanded configuration. In other examples, the rungs 26 may have a length between 1 mm and 5 mm. In still other examples, the rungs 26 may have a length between 2 mm and 3 mm.

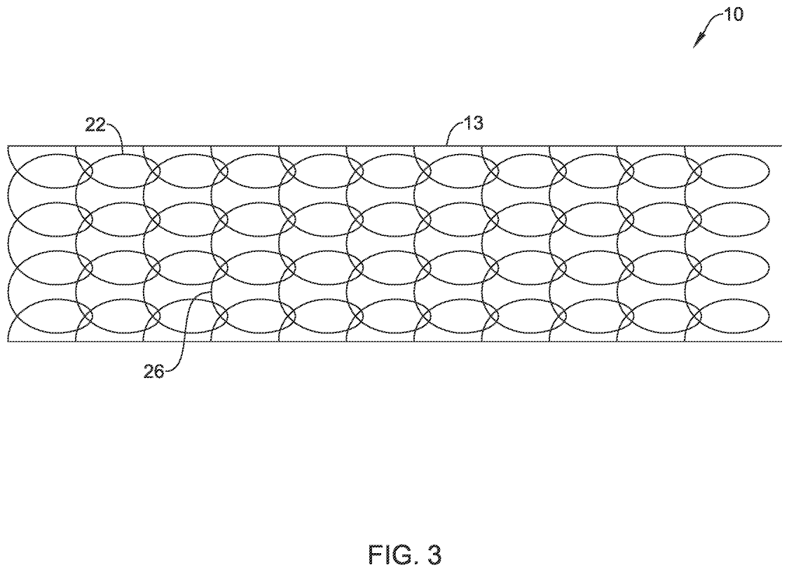

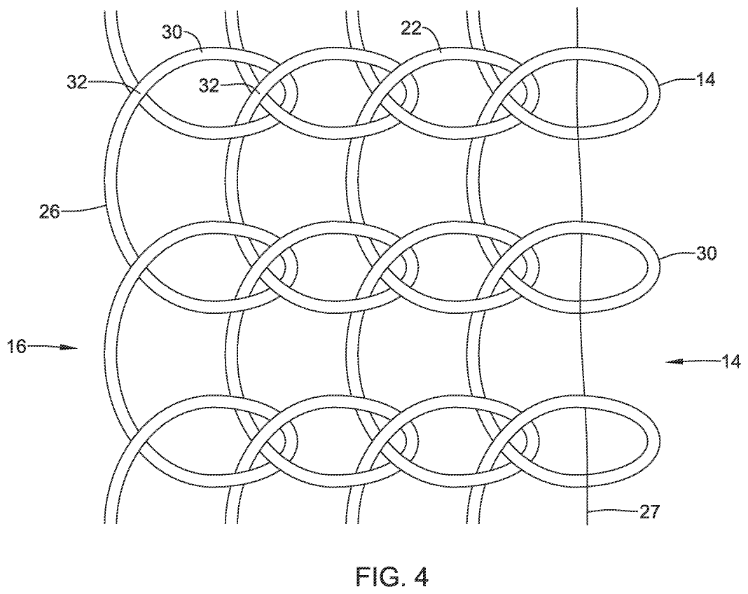

FIG. 3 illustrates the stent 10 in a radially constrained configuration disposed within a delivery sheath 13. When the stent 10 is radially collapsed and elongated as it is inserted into the delivery sheath 13, the helical interconnected twisted knit stitches 22 straighten into longitudinal columns, as shown in FIG. 3. The twisted knit stitches 22 elongate and the rungs 26 become shorter. The structure of the twisted knit stitches 22 in the radially collapsed, constrained configuration, is illustrated in FIG. 4. Each twisted knit stitch 22 may include a closed loop portion 30 and a crossed base region 32 defining a bottom of the closed loop. The loop portions 30 may be wrapped around the crossed base regions 32 of longitudinally adjacent twisted knit stitches 22. The crossed base regions 32 are distal of the loop portions 30, such that at the proximal end 16 of the stent, the crossed base regions 32 define an atraumatic structure, as shown in FIG. 4. While the loop portions 30 have an elongate or oval shape in the collapsed configuration shown in FIG. 4, the loop portions 30 may have a generally circular shape in the expanded configuration, as shown in FIG. 2. In some examples, the loops 30 may have a diameter of between 1 mm and 5 mm in the expanded configuration. In other examples, the loops 30 may have a diameter of between 2 mm and 3 mm.

The distal end 14 of the stent 10 may be defined by a series of free loop portions 30. In some embodiments, a first tether or suture 27 may be threaded through at least some of the free loop portions 30 at the distal end 14 and a second suture 27 may be coupled to the proximal end to facilitate elongation of the stent 10. The suture 27 coupled to the proximal end of the stent 10 may also be used for removing the stent, if so desired. The size of the free loop portions 30 at the proximal end may be increased or decreased to increase or decrease, respectively, the amount of tissue ingrowth at the proximal end achieved upon implantation of the stent 10.

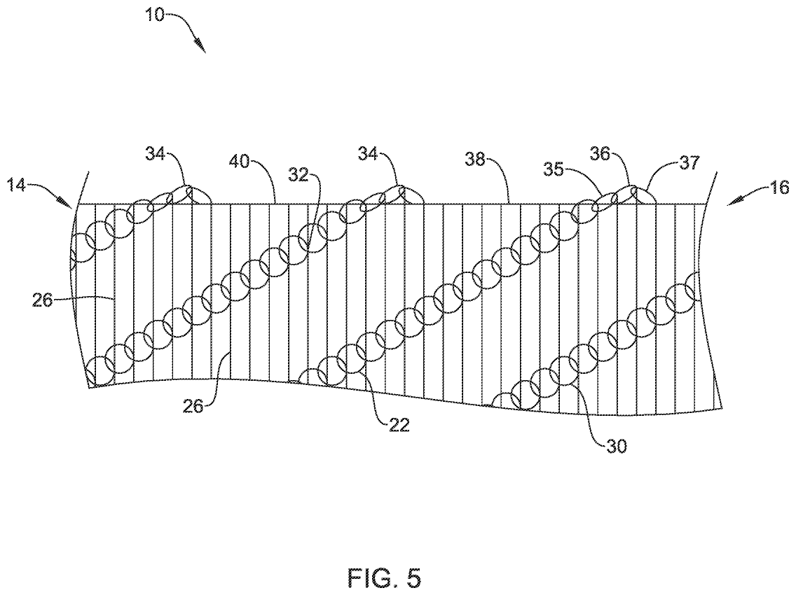

In the expanded configuration, the rungs 26 define an outer surface 40 of the stent 10 and the crossed base regions 32 of the twisted knit stitches 22 extend radially outward from the outer surface 40, as shown in FIG. 5. The crossed base regions 32 form a raised ridge 34 extending helically around the stent 10. In some examples, the raised helical ridge 34 may have a longitudinal cross-sectional wave shape, with a proximal facing slope 35, a crest 36, and a pocket 37 facing a proximal end 16 of the tubular member. In some examples, the crest 36 may protrude from the outer surface 40 between 0.5 mm and 5.0 mm. In a particular example, the crest 36 may protrude 1.5 mm from the outer surface 40. The distance is essentially the diameter of the loop portion 30, and the minimum distance is dependent on the diameter of the filament 24. For example, wire having a diameter of 0.003 inches to 0.014 inches (0.0762 mm to 0.3556 mm) may be used as the filament 24. In one example, a wire having a diameter of 0.006 inches (0.1524 mm) was used as the filament 24.

The space between the raised helical ridges 34 may define channels 38 extending between crests 36 of adjacent raised helical ridges 34. The channels 38 may provide a drainage feature for the stent 10. The raised helical ridges 34 may engage the tissue wall, while leaving at least a portion of the channels 38 spaced from the tissue wall, providing for drainage of fluid along the entire length of the stent 10. A covering or graft disposed over the stent or within the lumen may aid in defining the channels 38.

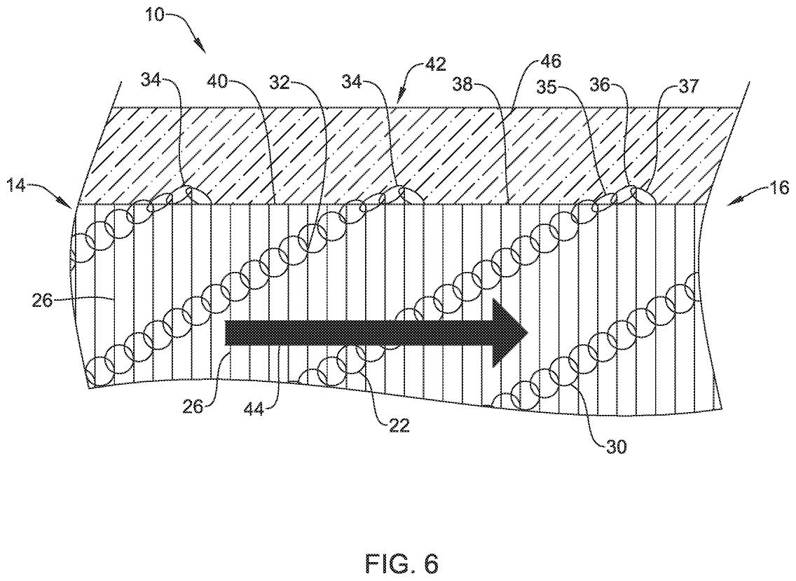

FIG. 6 illustrates the stent 10 disposed with a body lumen 42. The wave shape of the raised helical ridge 34 provides strong anti-migration properties in one direction and less in the opposite direction. The stent 10 may be loaded into a delivery sheath and placed in a body lumen in the preferred orientation to optimize resistance to the migration force on the stent, as shown in FIG. 6. This unique anti-migration feature may also provide a benefit during removal of the stent, as during removal the stent may be pulled in the direction with less anti-migration properties. This feature may make removal of the stent very easy for the physician without compromising any of the overall strong anti-migration properties of the stent 10.

When migration forces (arrow 44), such as peristalsis when the stent 10 is disposed within the esophagus or intestine, are exerted in a distal direction on the stent 10, the wave crest 36 provides resistance by pushing into the vessel wall 46, and the pocket 37 engages a portion of the vessel wall 46, as seen in FIG. 6, thereby preventing migration of the stent 10. The crest 36 is devoid of any sharp edge, barb, or quill. Rather, the crest 36 defines a smooth yet defined edge, as shown in FIG. 5. The anti-migration provided by the crest 36, is exhibited for each raised ridge 34 along the entire length of the stent 10. The wave shape of the raised helical ridge 34, in particular the gradual proximal facing slope 35, allows for removal of the stent 10 in the proximal direction without damage to the vessel wall 46.

The twisted knit stitches 22, and in particular, the loop portions 30 may be configured to match the level of tissue ingrowth desired and/or required. For example, increased tissue ingrowth may be achieved by increasing the number of loop portions 30 around the circumference of the stent 10. The pitch and/or angle of the helices may also be increased, and the size of the loop portions 30 may be altered. The configuration of the loop portions 30 may have a more pronounced effect on the tissue ingrowth in stents having a bare metal composition, devoid of any covering or graft.

The peristaltic motion in the esophagus and intestines occurs along the longitudinal surface of the vessel wall. Existing parallel knitted stents have raised loops in a straight formation along the entire length of the stent. The forces transferred to such stents by peristalsis is thus constantly exerted on the entire length of the stent. However, due to the helical ridges 34 of the stent 10, there is no direct transfer of force along the entire length of the stent. Instead, the vessel wall 46 exhibits force on the raised ridge 34 of the stent 10, but the force is intermittent, because no force is transferred to the outer surface 40 defined by the rungs 26 of the stent 10.

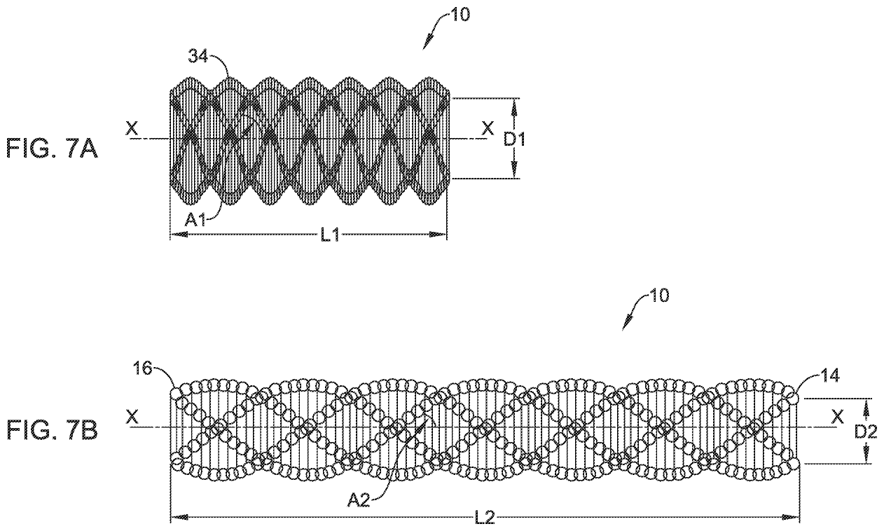

The configuration of the knit pattern as shown in FIG. 2, with a helical property may allow the stent to ‘store’ additional wire loops in a closed packed configuration that has a defined radial and axial flexibility. During deployment, as the stent 10 is released from the delivery sheath 13, the stent 10 may twist in a corkscrew manner as it relaxes and moves from the radially constrained delivery configuration in FIG. 3 to the radially expanded configuration of FIG. 7A. The distal end 14 and the proximal end 16 of the stent 10 may then be pushed or pulled to longitudinally stretch the stent 10 into the elongated configuration of FIG. 7B. This corkscrew twisting motion during deployment allows the stent 10 to increase in length without reducing in diameter, and may also help the stent 10 engage the walls of the body lumen in which the stent 10 is deployed. In particular, the raised helical ridges 34 of the stent 10 may engage the walls of the body lumen to secure the stent 10 in the elongated configuration, and prevent the stent 10 from retracting in length to the expanded configuration shown in FIG. 7A. In comparison, a conventional braid or a parallel knit stent structure may have a finite amount of material to operate and express their properties with. When conventional braided or parallel knit stents are elongated, the stent generally reduces in diameter to accommodate the change in length with the limited amount of material forming the stent structure. Conventional braided stents may accommodate a length change which results in a reduced diameter.

Elongation of the disclosed knitted pattern, as shown in FIG. 2, allows the stent 10 to change in length without a significant reduction in diameter, and also to maintain radial force as the excess material ‘stored’ in the design is available. As seen in FIG. 7A, in the radially expanded, relaxed configuration, the stent 10 has a series of helical ridges 34 of twisted knit stitches extending helically at a first angle of A1 relative to the longitudinal axis X-X. As the stent 10 elongates (e.g., by stretching the stent 10 from the radially expanded, relaxed configuration to a radially expanded, elongated configuration), the stent 10 may twist, and the angle of the helical ridges 34 relative to the longitudinal axis X-X may decrease to a second angle A2, as shown in FIG. 7B. FIGS. 7A and 7B demonstrate the disclosed knitted pattern in the stent 10 as it moves between a radially expanded, and longitudinally relaxed state (FIG. 7A) to a radially expanded, longitudinally elongated state (FIG. 7B).

FIGS. 7A and 7B illustrate the knitted stent 10 accommodating a change in length as the stent 10 is stretched or elongated from an initial length L1 in the radially expanded, relaxed configuration to an elongated length L2 is the radially expanded, elongated configuration. For example, the stent 10 may transition from an initial length L1 and inner diameter D1 in FIG. 7A to a length L2 while maintaining a substantially constant inner diameter D2 (D2 being equal or substantially equal to D1), as shown in FIG. 7B. Substantially constant or substantially equal is intended to mean the inner and/or the outer diameter of the tubular member forming the stent changes by less than or equal to 10% when stretched or elongated while in the radially expanded configuration. In some instances, the elongated length L2 may be 150% or more, 175% or more, 200% or more, 250% or more, or 300% or more of the initial length L1 while the diameter remains substantially constant. In some instances, the initial length L1 may be 50 mm to 60 mm and the elongated length may be 100 mm to 160 mm (thus elongating by 100% or more of the initial length L1), while the inner diameter D1/D2 remains constant at about 20 mm (20 mm±2 mm). The ability of the stent 10 to be elongated to such a large extent means the single size of stent 10 may be used in place of multiple current stent sizes of various lengths, such as elongated lengths of 60 mm, 80 mm, 100 mm, 120 mm, and 150 mm, for example. In this manner, the stent 10 may reduce the amount of inventory required to cover a wide range of stents needed for various medical procedures.

Another stent 10 with a length profile at the larger end of the spectrum often medically desired may be provided, such as a stent with a first length L1 of 120 mm in a radially expanded, relaxed configuration and an elongated (or stretched) length L2 of 300 mm in a radially expanded, elongated configuration, where the stent may have a constant inner diameter in both configurations. Providing the stent 10 in multiple diameters would further increase the variety of sizes covered by only a few stent sizes.

The inner diameter D1/D2 may be defined by an inner surface of the rungs 26. The stent 10 may thus have a first longitudinal length L1 and a first inner diameter D1 in the radially expanded, axially relaxed configuration (FIG. 7A) and a second longitudinal length L2 and a second inner diameter D2 in the radially expanded, axially elongated or stretched configuration (FIG. 7B), where the first longitudinal length L1 is less than the second longitudinal length L2, while the first and second inner diameters D1, D2 may be substantially the same. The stent 10 may be made in accordance with the methods described in US Publication No. 2020/0214858 A1, the entirety of which is incorporated herein by reference.

FIGS. 7A and 7B illustrate the great elongation ability of the stent 10. The design of the helical series of interconnected twisted knit stitches 22 allows the stent 10 to elongate without reducing in diameter, and also to conform to bends in a vessel without kinking. The increased conformability of the helical stent 10 is due to the ability of the circular loop knit design to elongate and compress at lower forces than the conventional knitted stents with parallel knit stitches.

The elongation characteristics of the stent 10 may allow the physician to vary the length of the stent in situ while maintaining the expanded diameter and the radial force of the stent constant. Namely, the stent 10 may be radially expanded in a body lumen, and thereafter, medical personnel may elongate or stretch the expanded stent 10 to a desired elongated length from its initial length when first expanded in the body lumen.

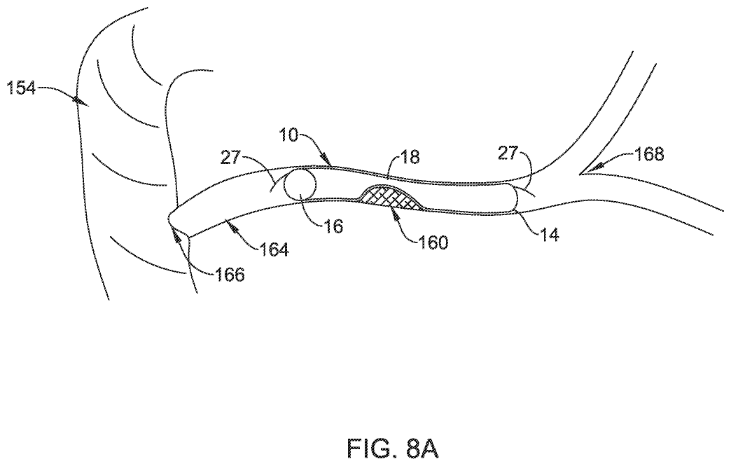

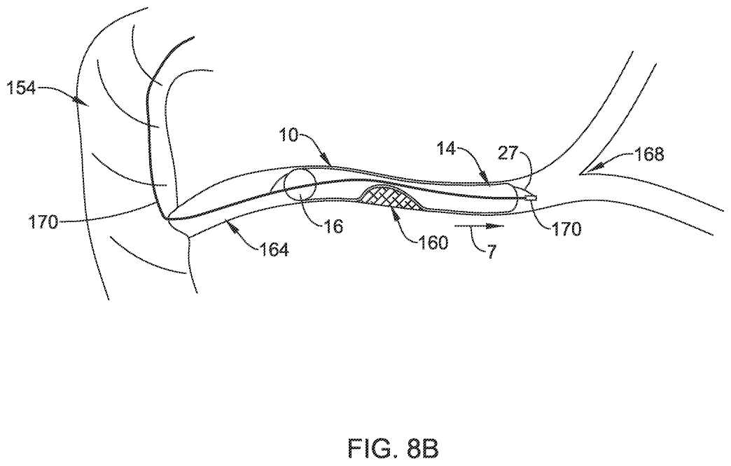

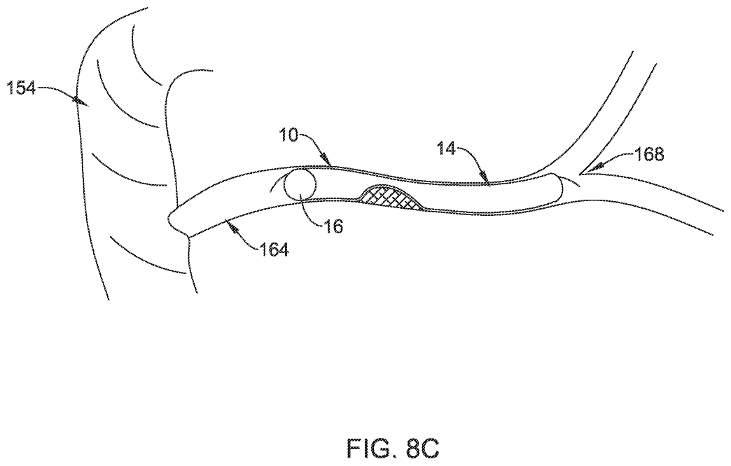

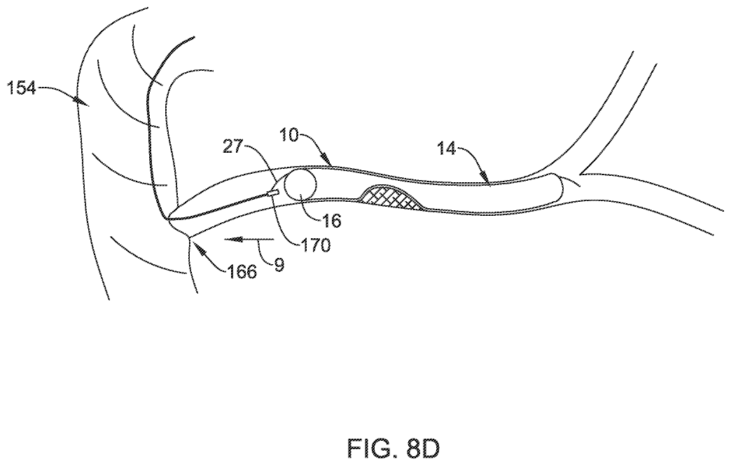

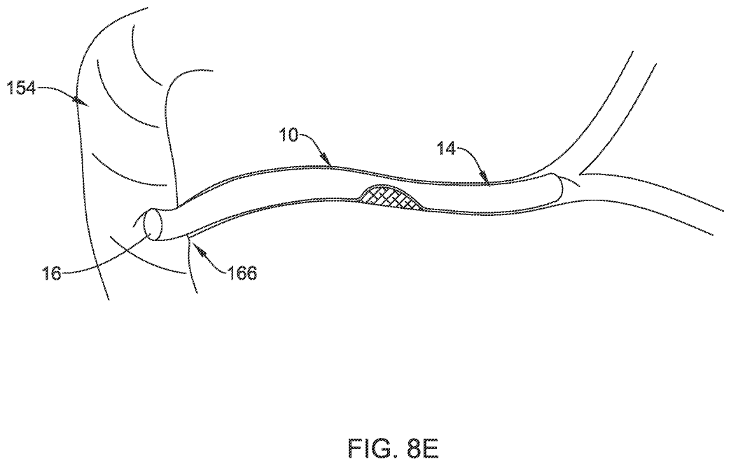

FIGS. 8A-8E illustrate the deployment of the stent 10 within the bile duct 164 and duodenum 154. The stent 10 may be delivered and expanded in the desired location, e.g. with the central region 18 of the stent disposed across a stricture 160 in the bile duct 164, as shown in FIG. 8A. The proximal end 16 of the stent may be oriented toward the ampulla 166 and the distal end 14 of the stent 10 may be oriented toward biliary/hilar branch 168. The physician may adjust the length of the stent 10 to the desired dimension based on the requirements of the patient's anatomy and position of the stricture 160. For example, for a bile duct stricture, the physician may wish to orient the distal end 14 of the stent so that it doesn't block the gall bladder or hilar branches, and to orient the proximal end of the stent within the duct or duodenum 154. As shown in FIG. 8B, after the stent 10 is advanced across the stricture 160 and radially expanded, the physician may track a grasping instrument or forceps 170 through the lumen of the stent 10 to the distal end 14 of the stent 10. The physician may then increase the length of the stent 10 (e.g., stretch the stent 10) by grasping the distal end 14 of the stent 10 and pulling the stent 10 distally, as indicated by arrow 7, further into the bile duct 164 to the desired position, such as just short of the biliary/hilar branch 168, as shown in FIG. 8C. In some embodiments, instead of grasping the expandable framework of the stent 10, the suture 27 attached to the distal end 14 of the stent 10 may be grasped with the forceps or other grasping instrument and pulled distally to elongate the stent 10. The proximal end 16 of the stent 10 may remain stationary as the distal end 14 is pulled distally, thus elongating the stent 10 with the stent 10 in a radially expanded configuration. As shown in FIG. 8D, the physician may additionally or alternatively use the grasper or forceps 170 to grab the proximal end 16 of the expandable framework of the stent 10 or the suture 27 attached to the proximal end 16 of the stent 10 to pull the proximal end 16 of the stent 10 proximally, as indicated by arrow 9, thus elongating the stent 10 with the stent 10 in a radially expanded configuration. Once the proximal end 16 of the stent 10 is at the desired position, for example when the proximal end 16 just protrudes through the ampulla 166 into the duodenum 154, the grasper/forceps 170 may be released from the stent 10 and be withdrawn, as shown in FIG. 8E.

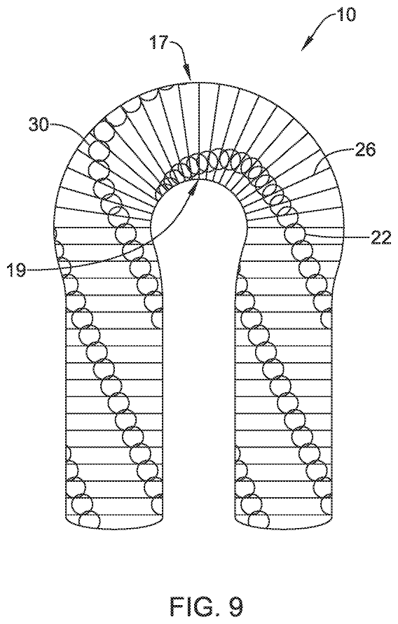

In addition to the biliary tract, the flexibility of the stent 10 may provide advantages for use in the enteral anatomy. As shown in FIG. 9, the structure of the interconnected twisted knit stitches 22 allows the stent 10 to bend up to 180 degrees without collapsing or kinking. The loop portions 30 defining the outside curve 17 of the bend elongate as a longitudinal spacing between longitudinally adjacent rungs 26 increases, and the loop portions 30 defining the inside curve 19 of the bend overlap one another more as the longitudinal spacing between longitudinally adjacent rungs 26 decreases. The outside curve 17 expands under small tension forces and the inside curve 19 compresses under small compression forces on the smaller radial surface. This combination of loop portions 30 elongating and compressing allows the stent 10 to bend without kinking at the inside curve 19.

Stenting of the colonic flexures with conventional stents has been recognized as problematic due to issues in correct stent placement. A conventional stent, if placed incorrectly, may be susceptible to migration due to non-symmetry of the stent across the stricture or the use of a stent that is too short and offers insufficient scaffold on one or both sides of the stent. Additionally, stent end stenosis often occurs with conventional stents placed in these regions as the stent is placed at an approximately 90-degree angle which may cause the stent to attempt to straighten out, causing abrasion of the contacting vessel wall. The ability of the physician to vary the length of the stent 10 without compromising the radial characteristics of the stent 10 makes the stent 10 particularly suitable for use in these locations with an improved outcome. The stent 10 is very flexible, which also is beneficial for this application.

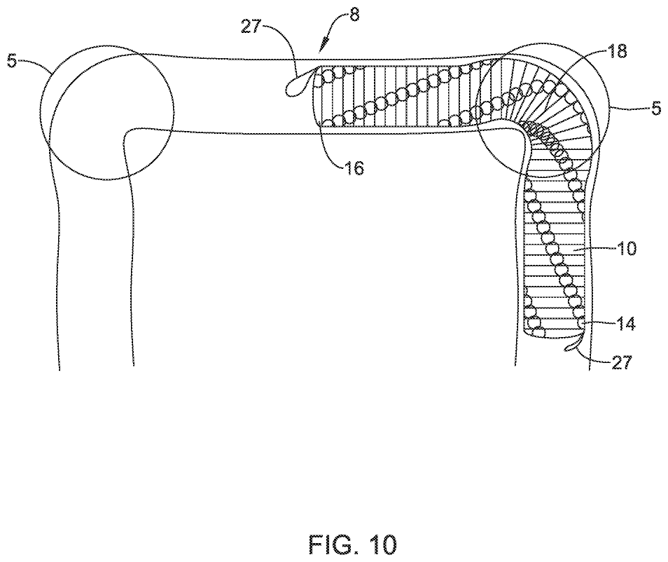

As shown in FIG. 10, the stent 10 may be deployed and radially expanded with the central region 18 disposed across a flexure 5. Similar to the deployment in the biliary tract described above, a forceps or other grasping instrument may be inserted through the stent 10 to grasp a portion of the stent 10, such as the suture 27 fixed to the distal end 14 of the stent 10 and pull the distal end 14 distally to a desired position, thereby elongating the stent 10. The forceps or other grasping device may additional or alternatively be positioned to grasp a portion of the stent 10, such as the suture 27 fixed to the proximal end 16 of the stent 10 and pull the proximal end 16 proximally to a desired position, thereby elongating the stent 10. The flexibility of the stent 10 allows for a more than 90-degree bend without kinking at the bend, making the stent 10 desirable for use in treating colonic flexures.

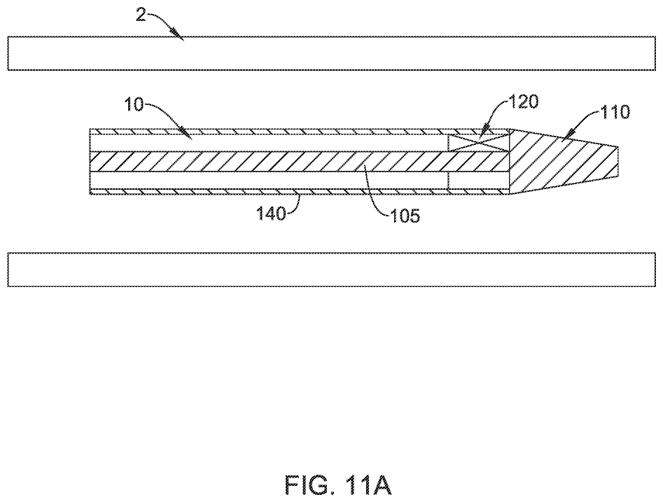

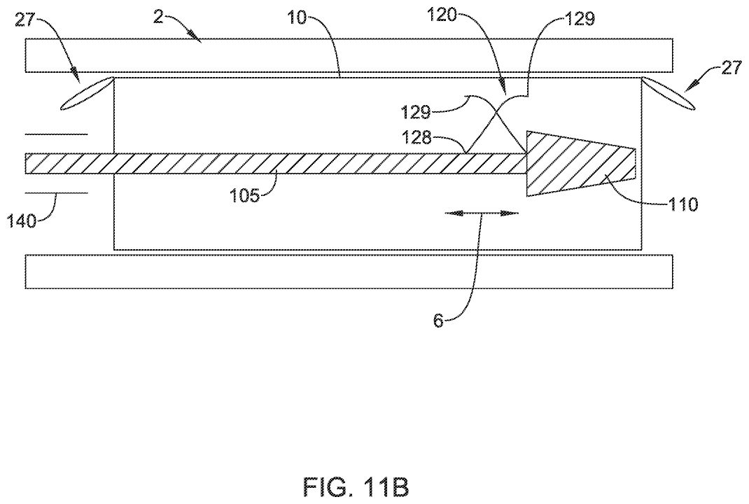

As an alternative to the grasper or forceps 170 used to elongate the stent, another grasping instrument is incorporated into a stent delivery system. The stent delivery system includes an engagement element configured to engage the sutures, or other structure, on the distal and/or proximal ends of the stent 10, as illustrated in FIGS. 11A and 11B. The delivery system may include an inner shaft 105 with a distal tip 110 and at least one capture element 120 mounted on a distal end region of the inner shaft 105 proximal of the distal tip 110, and an outer sleeve 140 surrounding the inner shaft 105 and slidable relative to the inner shaft 105. The outer sleeve 140, when surrounding the stent 10, may radially compress the stent 10 around a stent retention portion of the inner shaft 105. The distal end of the stent 10 may be positioned proximal of the capture element 120 when the stent 10 is radially compressed around the stent retention portion by the outer sleeve 140. FIG. 11A shows the delivery system disposed within a body lumen 2. In some instances, the capture element 120 may be actuatable between a first configuration in which the capture element 120 is constrained within the outer sleeve 140 and a second configuration in which the outer sleeve 140 is withdrawn proximal of the capture element 120. For example, the capture element 120 may be configured to move between a first, radially collapsed configuration positioned adjacent the inner shaft 105 when constrained within the outer sleeve 140, as shown in FIG. 11A, and a second, radially extended configuration in which the capture element 120 extends radially outward from the inner shaft 105 when the outer sleeve 140 is withdrawn proximally, as shown in FIG. 11B. The capture element 120 may be configured to move to the second configuration automatically when the outer sleeve 140 is retracted. The capture element 120 may be configured to extend radially a sufficient distance such that it may engage the suture 27 coupled to the stent 10, or other structure of the stent 10, when the stent 10 is in the radially expanded configuration. In some embodiments, the capture element 120 may extend radially outward 3 mm or more, 5 mm or more, or 10 mm or more from the outer surface of the inner shaft 105 in the radially extended configuration. The inner shaft 105 may be moved distally and proximally, as indicated by arrow 6, to move the capture element 120 into engagement with the suture 27 on the distal or proximal end of the stent 10 to elongate the stent 10, as discussed above. After elongation of the stent 10 by stretching the distal end and/or the proximal end of the stent 10, the outer sleeve 140 may be reverted back over capture element 120 (i.e., either by withdrawing the inner shaft 105 and the capture element 120 into the lumen of the outer sleeve 140 or advancing the outer sleeve 140 distally over the capture element 120) to re-constrain the capture element 120 within the outer sleeve 140, followed by removal of the delivery device.

The capture element 120 may include any desired structure configured to engage the suture 27 and/or other structure of the stent 10. In some embodiments, the capture element 120 may include at least one hook 129. In one embodiment, the capture element 120 includes both a first distally facing hook 129 and a second proximally facing hook 129, as shown in FIG. 11B. The hooks may be rounded and atraumatic to avoid injury to the tissue. The capture element 120 may be made of metal or polymer, and may be sufficiently rigid to maintain its shape when engaged with the suture 27 and pushed or pulled to elongate the stent 10. A portion of the capture element 120, such as the connection 128 with the inner shaft 105, may be formed from a flexible material, such as thin metal or a polymer, configured to bend or flex. In some embodiments, the capture element 120 may be biased in the radially extended position. For example, the capture element 120 may be made of a shape memory material and/or a super-elastic material, such as nitinol. In other examples, the connection 128 may be a hinged connection between the capture element 120 and the inner shaft 105, with the capture element 120 being prevented from moving beyond the first and second configurations.

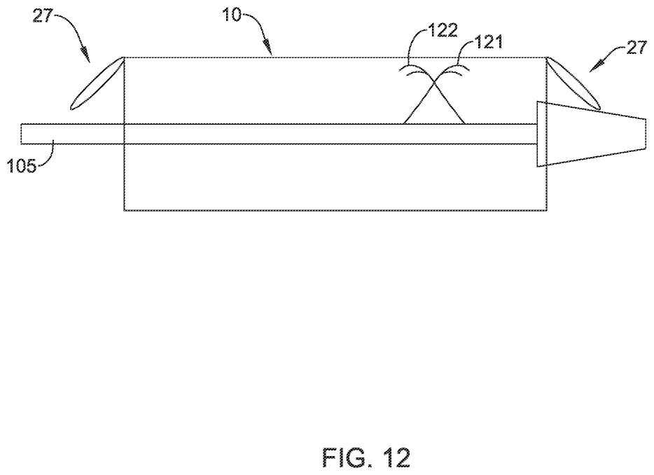

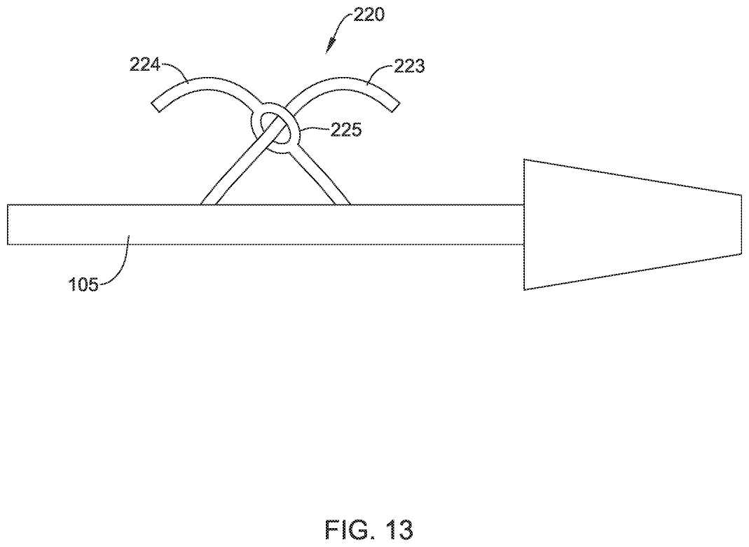

In other embodiments, the capture element 120 may include a plurality of distally facing hooks 121 and/or a plurality of proximally facing hooks 122, as shown in FIG. 12. In a further embodiment, the capture element 220 may include a first hook 223 coupled to a second hook 224, as shown in FIG. 13. One of the first and second hooks may include an opening (e.g., an eyelet, slit, slot, etc.) through which the other of the first and second hook may moveably extend through. For example, the second hook 224 may be a proximally facing hook and may include an opening 225 through which the first hook 223 extends, and the first hook 223 may be a distally facing hook. In other embodiments, the first hook 223 and the second hook 224 may each include a plurality of hooks, similar to FIG. 12. The coupling may provide support for the capture element 220 as it engages and pushes or pulls the sutures to elongate the stent 10. The delivery device with a capture element 120 provides the benefit of a “one-for-all device”, eliminating the need for additional grasping devices to be inserted to the stent location.

The stents, delivery systems, and the various components thereof, as described above, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic Nitinol; other nickel alloys such as nickel-chromium-molybdenum alloys, nickel-copper alloys, nickel-cobalt-chromium-molybdenum alloys, nickel-molybdenum alloys, other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys; platinum enriched stainless steel; titanium; combinations thereof; and the like; or any other suitable material.

Some examples of suitable polymers for the stents or delivery systems may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In at least some embodiments, portions or all of the stents or delivery systems may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent configured to change in length while maintaining a constant inner diameter, the stent comprising:
a tubular member having a proximal end, a distal end, and a longitudinal axis extending therebetween, the tubular member comprising a knitted filament forming a plurality of twisted knit stitches with rungs extending circumferentially between adjacent twisted knit stitches, wherein each twisted knit stitch is interconnected with a longitudinally adjacent twisted knit stitch forming a series of linked stitches, the tubular member configured to automatically radially expand from a constrained configuration during delivery to a radially expanded configuration, wherein when in the radially expanded configuration, the tubular member has a first length and a first inner diameter and is configured to be stretched to an elongated configuration having a second length and a second inner diameter; and
wherein the first length is shorter than the second length and the first and second inner diameters are substantially the same.

2. The stent of claim 1, wherein the second length is at least 200% or more of the first length.

3. The stent of claim 1, wherein the first length is about 50 mm to about 60 mm and the second length is about 100 mm to about 160 mm.

4. The stent of claim 1, wherein when in the radially expanded configurations the series of linked stitches defines a helix, the helix having a first angle relative to the longitudinal axis when the tubular member is at the first length and a second angle relative to the longitudinal axis when the tubular member is at the second length, wherein the first angle is larger than the second angle.

5. The stent of claim 1, wherein when in the constrained configuration for delivery, the series of linked stitches defines longitudinal columns.

6. The stent of claim 1, wherein each of the plurality of twisted knit stitches includes a loop portion and a crossed base region.

7. The stent of claim 6, wherein each of the plurality of twisted knit stitches is formed by a single filament defining the loop portion and the crossed base region.

8. The stent of claim 6, wherein the loop portion of at least some of the twisted knit stitches is wrapped around the crossed base region of the longitudinally adjacent twisted knit stitch.

9. The stent of claim 1, further comprising a first suture threaded through at least some of the twisted knit stitches at the distal end and a second suture threaded through at least some of the twisted knit stitches at the proximal end of the tubular member.

10. A stent assembly comprising:
a stent having a proximal end, a distal end, and a longitudinal axis extending therebetween, the stent comprising a knitted filament forming a plurality of twisted knit stitches with rungs extending circumferentially between radially adjacent twisted knit stitches, wherein each twisted knit stitch is interconnected with a longitudinally adjacent twisted knit stitch forming a series of linked stitches, the stent configured to automatically radially expand from a constrained configuration during delivery to a radially expanded configuration, wherein when in the radially expanded configuration, the stent has a first length and a first inner diameter and is configured to be stretched to an elongated configuration having a second length and a second inner diameter, wherein the first length is shorter than the second length and the first and second inner diameters are substantially the same; and
a delivery device including an outer sleeve and an inner shaft slidable within the outer sleeve, the inner shaft having a distal tip and at least one capture element, wherein the at least one capture element is configured to move between a first configuration positioned adjacent the inner shaft when constrained within the outer sleeve, and a second configuration extending radially outward from the inner shaft when released from the outer sleeve.

11. The stent assembly of claim 10, wherein the at least one capture element includes at least one hook.

12. The stent assembly of claim 11, wherein the at least one hook includes a first distally facing hook and a second proximally facing hook.

13. The stent assembly of claim 12, wherein the at least one capture element includes a plurality of distally facing hooks and a plurality of proximally facing hooks.

14. The stent assembly of claim 11, wherein the at least one hook includes a first hook coupled to a second hook.

15. The stent assembly of claim 14, wherein the first hook extends through an opening in the second hook.

16. The stent assembly of claim 10, wherein the at least one capture element is biased in the second configuration.

17. The stent assembly of claim 10, wherein the at least one capture element extends radially 5 mm or more from an outer surface of the inner shaft in the second configuration.

18. A method of supporting a body lumen at a stricture, comprising:
delivering a stent within the body lumen with a central region of the stent disposed across the stricture;
radially expanding the stent to a radially expanded configuration in the body lumen, wherein the stent includes a tubular member having a distal end and a proximal end and a longitudinal axis extending therebetween, the tubular member comprising a knitted filament forming a plurality of twisted knit stitches with rungs extending circumferentially between adjacent twisted knit stitches, wherein each twisted knit stitch is interconnected with a longitudinally adjacent twisted knit stitch forming a series of linked stitches, the tubular member having a first length and a first inner diameter in the radially expanded configuration; and
thereafter, stretching the stent within the body lumen to a radially expanded and elongated configuration having a second length, wherein the second length is greater than the first length.

19. The method of claim 18, wherein the stent has a second inner diameter in the radially expanded and elongated configuration, wherein the second inner diameter is substantially the same as the first inner diameter.

20. The method of claim 18, wherein the second length is at least 200% or more of the first length.
